(19) United States
(12) Patent Application Publication
(10) Pub. No.: US 2016/0355411 A1
(43) Pub. Date: Dec. 8, 2016

(54) PORTABLE WATER TREATMENT SYSTEM USING PRECISE ENERGY SEPARATION

(71) Applicant: Fahs Stagemyer, LLC, Woodstock, CT (US)

(72) Inventor: Richard W. Fahs, II, Woodstock, CT (US)

(21) Appl. No.: 15/153,538

(22) Filed: May 12, 2016

Related U.S. Application Data
(60) Provisional application No. 62/160,315, filed on May 12, 2015.

(51) Int. Cl. C02F 1/32; C02F 1/00; B01D 46/00; B01D 53/00; B01D 53/86; A61L 2/10; A61L 2/08; A61L 2/26; A61L 9/20; H01G 11/10; H01G 11/32; H01G 11/52; H01G 11/58; B08B 7/00; B08B 7/02; H02J 7/00; C02F 1/72

(52) U.S. Cl. CPC C02F 1/325; C02F 1/725; C02F 1/001; B01D 46/0028; B01D 53/007; B01D 53/86; A61L 2/10; A61L 2/088; A61L 2/26; A61L 9/20; A61L 9/205; H01G 11/10; H01G 11/32; H01G 11/52; H01G 11/58; B08B 7/0035; B08B 7/0057; B08B 7/026; H02J 7/0024; C02F 2305/10; C02F 2201/008; C02F 2201/009; C02F 2201/326; B01D 2255/802; B01D 2259/804; A61L 2202/11; A61L 2202/13; A61L 2209/12; H02J 7/345

(57) ABSTRACT

A portable system for treatment of liquids, gases, or both using precise energy separation (PES) is described herein. The system includes a power generation component which charges one or more energy storage units and powers the PES component of the portable system. The PES component includes one or more energy of dissociation sources and the energy storage units power the sources to provide an effective amount, intensity, and frequency of a promoter energy to specifically dissociate one or more target bonds of the target molecule present in contaminated liquids, gases, or both. Optionally, the energy stored in the system can act as a supplementary or back up power source.

### CROSS-REFERENCE TO RELATED APPLICATION

This application claims benefit of and priority to U.S. Provisional Application Ser. No. 62/160,315 filed on May 12, 2015, which is incorporated herein in its entirety.

### FIELD OF THE INVENTION

The present invention relates to the treatment and remediation of liquids, gases, or both in a portable system using precise energy separation.

### BACKGROUND OF THE INVENTION

Water contamination represents a global problem as water containing a variety of contaminants create significant environmental issues and health hazards. Wastewater may be contaminated with chemical and/or biological pollutants which are not considered environmentally safe and render the water unsafe for drinking, washing or even agriculture. Furthermore, air contaminated with chemicals and/or particulate matter also creates significant environmental issues and health hazards.

As an example, in some regions of the world, access to potable water may be limited or non-existent. Overall, it has been estimated that approximately 11% of the world population does not have access to safe water. Thus, in order to attain potable water in such remote regions it is desirable to provide inexpensive and preferably portable means of treating contaminated water found in the region to purify/sterilize the water. As electrical grids in certain areas and regions of the world may be otherwise unavailable or non-dependable (i.e., prone to interruption or failure) it is also desirable to provide a means for treating contaminated water which includes an independent power source, preferably not requiring electricity or a gasoline or diesel burning generator.

It is therefore an object of the invention to provide an independent and portable system that can be used for the remediation of liquids and/or gases.

It is also an object of the invention to provide such a system wherein various power generation sources may be used and the energy generated can be stored.

It is a further object of the invention to provide a system wherein stored power in an energy storage component can be optionally used as a source of supplementary or back up power when not in use for purifying water.

### SUMMARY OF THE INVENTION

A system which can be used for the remediation of liquids, such as water, wastewater, and/or contaminated water, and/or gases, such as contaminated and/or polluted air using a precise energy separation (PES) component and a power generation component is described herein. Such a system is particularly advantageous for humanitarian or environmental applications where a means for remediating water in remote regions of the planet and can be readily used to provide access to safe, clean, potable and drinkable water. Such a system is also particularly advantageous for humanitarian or environmental applications where a means for remediating air in remote regions of the planet and can be readily used to provide access to purified air.

The system contains a power generation component configured and coupled to a plurality of energy storage units which are charged by one or more power generation sources present in the component. Preferably the power is supplied by a hand crank, animal or bicycle, or a motor, or is connected to an electrical source. Output from the storage units delivers stored energy to one or more energy of dissociation sources present in a PES component. A control module is used to direct energy to storage or to the PES component for sterilization. The PES is used to direct sterilizing energy into a liquid or gas storage tank (or other liquid, such as milk), in an amount and at a wavelength and frequency effective to kill bacteria, viruses and other contaminants in the liquid. Optionally, the output may be configured and coupled to deliver energy to power other systems such as a cell phone or light source, when the PES component is not in use. In some embodiments, the energy storage units may include means to couple the storage units to at least one external power source, such as a local electrical grid system, which can be used to charge the plurality of energy storage units.

A variety of power generation sources can be used to charge the energy storage units, such as a dynamo, solar panels, turbines, fuel cells, optical rectennas, hydroelectric system, electrical generator, pulse generator, as well as the sources listed above. In a preferred portable embodiment, a hand cranked dynamo is used to generate power. In other embodiments, the dynamo generates energy/power by spinning motion when rotated by a person on a bicycle or vehicle or an animal, a water or wind driven turbine, or is rotated by any other suitable means. A dynamo may also be incorporated into a vehicle, such as a cart or a bicycle, as part of a wheel component such that motion of the vehicle rotates the dynamo and energy/power is generated.

The control module controls the charging and discharging process of the units. In preferred embodiments, the energy storage units include one or more supercapacitors or ultracapacitor cells, or less preferably other types of energy storage units, such as batteries. The super- or ultracapacitor cells are electrochemical double layer capacitors (EDLCs) that can store energy and have high capacitance as compared to traditional capacitors. Super- or ultracapacitor cells may additionally have pseudocapacitive properties to improve their storage capacity. The use of supercapacitors or ultracapacitor cells in the energy storage units allows for excellent power delivery capabilities to energy of dissociation sources that may require high voltages and/or high pulsed voltages.

In a preferred embodiment, the one or more power generation components are configured and coupled to charge the plurality of energy storage units, which when at least partially or fully charged can be discharged and used to power energy of dissociation source(s) of a PES system (i.e., xenon lamp or tunable laser) present in the system. The energy storage units upon discharge provide power to one or more outputs which are configured and coupled to the PES component and power the energy of dissociation source(s). In certain embodiments, the plurality of supercapacitors or ultracapacitors of the system can be charged in parallel and discharged in series to effectively multiply the voltage and produce a high voltage discharge pulse. In such an embodiment, the energy storage units containing a plurality of super- or ultracapacitor cells effectively function as a Marx generator. In such a pulse generating system, the control module is used to control the charge and discharge cycle(s) of the super- or ultracapacitor cells needed to provide the appropriate voltage and/or voltage pulse required.

In some embodiments, the system contains two or more separate banks of energy storage units and each bank contains a plurality of super- or ultracapacitor cells. In a preferred embodiment, at least one energy capacitor-based storage bank can be charging, while at least one bank is in use and thereby in active discharge to supply power to a load or source, and any of the remaining partially or fully charged energy banks can be in stand-by reserve mode, at any given time.

### BRIEF DESCRIPTION OF THE DRAWINGS

FIG. 1 shows a non-limiting example of a water treatment system as described herein. The system 10 includes a power generation component (100), energy storage component (200), PES component (300), and energy of dissociation source (400) and the components are housed in a main chamber/housing body (500).

FIG. 2 shows a non-limiting example of a water treatment system 20 including a hand cranked dynamo (110), a xenon lamp energy of dissociation source (410), and a spigot/faucet component (510) present as part of the main chamber/housing body (500) of the system.

### DETAILED DESCRIPTION OF THE INVENTION

### I. Definitions and Mechanisms

“Water”, as used herein refers to any solution that has water as a primary component and includes one or more chemical or biological contaminants. The aforementioned solution may also contain macroscopic contaminants, such as sediment. Contaminated water may come from any source including, but not limited to wells, cisterns, lakes, rivers, holding tanks, rain water, and water discharged from industrial sources.

“Purer water”, or “pure water”, as used herein, refers to water from which some or all contaminants have been removed. It is understood that since what constitutes a contaminant in water depends on what is subjectively considered undesirable, pure water herein can refer to water that includes solutes and other materials not considered contaminants in the context at hand.

“Purer liquid”, or “pure liquid”, as used herein, refers to one or more liquids from which some or all contaminants have been removed. It is understood that since what constitutes a contaminant in a liquid depends on what is subjectively considered undesirable, pure liquid herein can refer to water that includes solutes and other materials not considered contaminants in the context at hand.

“Purer gas”, or “pure gas”, as used herein, refers to one or more gases from which some or all contaminants have been removed. It is understood that since what constitutes a contaminant in a gas depends on what is subjectively considered undesirable, pure gas herein can refer to gas that includes particulates and other materials not considered contaminants in the context at hand.

“Supplementary or backup power,” are terms which are used interchangeably and refer to power which can be supplied to replace power usually generated by another separate power source when that power source is otherwise unavailable due to a failure or outage, or is otherwise unavailable for other reasons, such as requiring maintenance.

“Banks of energy storage units,” as used herein, refer to two or more separate banks which are each comprised of one or more energy storage units.

“Biological contaminant,” as used herein, refers to an undesirable contaminant of biological origin. The term “biological contaminant” encompasses biomolecules, such as proteins, polysaccharides, and polynucleotides, infections agents, such as viruses, as well as single and multi-celled organisms, such as bacteria, protozoa, plankton.

“Bond dissociation energy,” as used herein, refers to the standard enthalpy change when a bond is homolytically cleaved.

“Bond energy,” as used herein, refers to the average of the sum of the bond dissociation energies in a molecule.

“Component products,” as used herein, refers to known ions or atoms composed of only elements found within the target molecule. Individual component products have a chemical formula distinct from the target molecule. An example is N₂ and H₂, which are each component products of NH₃.

“Configured to,” as used herein, refers to the design, arrangement, or set up of a system and selection of components and subcomponents as necessary and/or required to perform a particular or given function, such as for example delivering power to an output for delivery to another system or for receiving power from an input coupled to a power source(s).

“Coupled to,” as used herein, refers to directly coupling (i.e. connecting) one element (i.e. output) of a system or component to another element (i.e. input) by any suitable means available, such as through a wiring system. Optionally, other intervening elements may also be present.

“Catalyst,” as used herein, refers to any chemical which enhances the rate and/or efficiency of molecular dissociation compared with the rate and/or efficiency of dissociation in the absence of the catalyst.

“Chemical waste,” as used herein, refers to any inorganic or organic substance, present in any physical state, that is unwanted in a given sample due to environmental or toxicity concerns.

“Dissociation,” as used herein, refers to breaking the bonds of a molecule. Dissociation in the current process is requires that the original bonds of the target molecule do not re-associate.

“Excited state,” as used herein, refers to a state in which one or more electrons of an atom or molecule are in a higher-energy level than ground state.

“Energy of dissociation source” as used herein refers to any device, apparatus, or combination thereof, which supplies the energy of dissociation energy required to dissociate target bonds within a target molecule. The energy of dissociation source must supply suitable intensity and suitable frequency for target bond dissociation. An example of an energy of dissociation source is an xenon lamp. An energy of dissociation source can optionally contain a catalyst. An example of such an energy of dissociation source is a titanium dioxide catalyst and a xenon lamp coupled to a pulse generator.

“Energy storage unit,” as used herein, refers to one or more units that can store power upon being charged and can be discharged to release the stored power upon discharge. Exemplary energy storage units may be selected from chemical, electrochemical, electrical, and thermal-based storage units. Preferred energy storage units include capacitance-based storage units such as supercapacitors or ultracapacitor cells.

“Irradiation,” as used herein, refers to exposing a sample to beams of particles or energy, such as a form of electromagnetic or acoustic radiation. In certain embodiments, irradiation involves exposing a sample to light.

“Marx generator,” as used herein, refers to a plurality of capacitors, such as super- or ultracapacitors, which can be charged in parallel and discharged in series to effectively multiply voltage and produce a high voltage discharge pulse.

“Non-target molecule,” as used herein, refers to the any substance within a sample containing target molecules which is not affected by the process.

“Power generation components,” as used herein, refer to any apparatus or system which can be used to generate energy. Examples of power generation components include, but are not limited to, a dynamo, solar panels, turbines, fuel cells, optical rectennas, hydroelectric system, electrical generator, pulse generator, and other suitable power generation systems.

“Promoter,” as used herein, refers to the energy required for dissociation of a target bond, which is both selective for the target bond and sufficient to prevent re-association of the bond.

“Energy of dissociation source,” as used herein, refers to any chemical, apparatus, or combination thereof, which supplies the energy of dissociation with the energy required to dissociate target bonds within a target molecule. The energy of dissociation source must supply suitable intensity and suitable frequency for target bond dissociation. An example of an energy of dissociation source is a xenon lamp coupled to a pulse generator. An energy of dissociation source can optionally contain a catalyst. An example of such an energy of dissociation source is a titanium dioxide catalyst and a xenon lamp coupled to a pulse generator.

“Precise Energy Separation (PES),” as used herein refers to the use of highly specific energy to selectively cleave specific bonds to dissociate a target molecule.

“Remediation,” as used herein, refers to treatment of liquids, such as wastewater or drinking water, and/or gases, such as air, to decrease the concentration of one or more undesirable contaminants. The contaminants can be biological or chemical contaminants.

“Sample” as generally used herein refers to at least one target molecule which is subjected to the dissociation process. A sample can comprise both target and non-target molecules.

“Stand-by reserve mode,” as used herein, refers to the state of an energy storage unit(s) or bank of energy storage units, wherein the energy storage unit is at least partially or more preferably fully charged and is not actively being charged or discharged. The unit can be discharged to release the reserve stored power as needed.

“Supercapacitors” or “ultracapacitors,” as used herein, are interchangeable terms that refer to electric double-layer capacitor cells, also known as electrochemical double layer capacitors (EDLCs). Supercapacitors store energy by physically separating positive and negative charges and have capacitance (preferably at least 100 F g⁻¹ or greater) and energy or power density values, which are significantly higher than those of traditional capacitors. Super- or ultracapacitors may be capacitive alone or may also have pseudocapacitive properties.

“Supplementary power,” as used herein, refers to power which can be supplied in addition to power generated by another separate power source.

“Target bond,” as used herein, refers to any bond within a target molecule. Target bonds can be covalent (including single and multiple covalent bonds), ionic, or “weak bonds” including dipole-dipole interactions, London dispersion forces, or hydrogen bonding. In certain embodiments, the target bonds are covalent bonds.

“Target molecule” as used herein refers to a molecule, or portion of a macromolecule, that contains at least one bond.

“Volatile organic compound” (VOC), as used herein, refers to organic compounds with high enough vapor pressure to evaporate and enter the atmosphere at ambient temperature and pressure. Examples of VOCs include low-molecular weight organic compounds such as alcohols, esters, ethers, aldehydes, thiols, and ketones.

### II. Methods of Use of Portable PES-Based Remediation System

The system described herein includes a PES component and can be used for remediation by irradiating water or other liquids, including but not limited to, milk containing one or more biological contaminants, chemical contaminants, or combination thereof with an amount of energy at a frequency and intensity effective to selectively break one or more bonds within one or more target molecules present in the contaminated liquid, such as water. The system described herein can also be used for remediation by irradiating one or more gases, including but not limited to air, containing one or more biological contaminants, chemical contaminants, or combination thereof with an amount of energy at a frequency and intensity effective to selectively break one or more bonds within one or more target molecules present in the contaminated gas, such as air. In preferred embodiments, the irradiation energy provided is in the form of light.

In certain embodiments of the system, the energy units of the system are charged by the one or more power generation components, such as a dynamo, and the stored energy is controllably delivered to one or more energy of dissociation source(s) present in the PES component. The energy of dissociation source can be a bulb or a lamp which provides pulsed light having a particular frequency and intensity (e.g., a nano- or picosecond burst of light) or a tunable laser which can be used to selectively induce the photo-dissociation of one or more target molecules and eliminate one or more contaminants present in the one or more liquids and/or gases.

In other embodiments, the one or more outputs of the energy storage units of the system can be optionally configured and coupled to provide power to external systems, such as a home's electrical system, lighting, cell phone or other individual devices (such as, for example, a motor), and the power stored in the energy storage units can be used as a source of power, or an alternate source of supplementary and/or back up power, as needed, preferably when the PES component is not in use.

The one or more outputs of the energy storage units of the system can provide stored electrical power to local and/or regional energy grids. Such energy storage units of the systems described herein represent efficient mobile or stationary PES mini-grid systems. Such a PES mini-grid system may be used for individual use or for a home or localized community. A PES mini-grid system can use any available energy, store the energy, such as in a supercapacitor, supercapacitor hybrid, or similar UPS unit as described above, so to provide ultra-fast burst of energy of DC power (or via a converter for AC power) for use in a home, car, airplane, ship/barge/boat, community, isolated and/or harsh location, handheld or carried device, propulsion, robots (including nanobots), heating ventilation air conditioning (HVAC), tractor trailers, carts, bicycles, motorbikes, drones, medical, 3-D printing (i.e., sintering, laser, heating-based) systems water-air and surface purification systems. PES-based systems, as described here, can replace the dependence on current electrical and fuel generated or similar energies, which limit mobility and quality of life and suffers from vulnerability of large utility systems, and in many cases where these systems are polluting energy systems.

The energy stored in energy storage units of the systems described herein can be a stationary or mobile PES mini-grid system. Such a mobile system can be carried and/or moved by a human or animal. This stored energy can be used by taking very small amounts of this stored energy (depleting very little of the total amount of stored energy and at the same time recharging (replenishing) the used portion of the stored energy—if desired) and by use of a pulse forming network (PFN) which can compress that small amount of stored or directly collected energy and can release very large amounts as energy bursts (such that this system can treat larger volumes of liquids and/or gases). These ultra-fast discharges of high intensity energy, can selectively (via coherent controlled via a pulse shape and/or a two to multi-photon process) dissociate target molecules in the gas, a liquid, and/or on a surface. This controlled discharge can create a plasma, electric field, and/or a pulsed wave of electromagnetic pulse or acoustic wave energy which can penetrate a solid. This energy discharge can create energy via a pulse generator, a plasma, and/or a fuel cell. The use of this form of stored energy and use of this stored energy via a PES process for water splitting and hydrogen production and collection is a more efficient means to split water, as compared to electrolysis. In some embodiments, this energy discharge can be used to clean and/or sterilize wounds. In some other embodiments, the energy discharge can be used to treat/purify gases, such as air, liquids, such as water, and/or surfaces. Without limitation, surfaces include, but are not limited to, the surfaces of solids, such as soil, items (i.e., desks, tables, household items), plants, fruits, vegetables, clothing, toys, personal healthcare items, tools, equipment, electronics, hospital/medical devices, hospital rooms/equipment, workspaces, tissues/wounds, surfaces of communal spaces; wherein the surfaces may be porous or non-porous. The surfaces may be contaminated by chemical and/or biological contaminants. Energy discharge, in the form of a plasma and/or an EMP, can produce an electrical field and/or an acoustic wave which can be used as a power source for robots or to activate any controlled molecular dissociation or re-association of target bonds without the formation of unwanted by-products. This PES system can be used to promote energy production via water splitting, electron capture, ignition of a fuel, such as hydrogenated nickel or similar material, or an energy or activation process for a metamaterial for use as a heat source, cooling source, for medical or any needed energy end use.

In the case of liquids or gases, such as water and/or air, containing a chemical contaminant, the target molecule is typically a chemical pollutant, such as volatile organic compound, present in the liquid and/or gas. In order to eliminate a chemical contaminant from a water and/or air sample, energy is introduced at a frequency and intensity to selectively dissociate one or more target bonds in the target molecule, causing the target molecule to dissociate into component products compositionally distinct from the target molecule. Generally, the bonds of the target molecule do not reform because the components are no longer reactive with each other. The process does not produce component products by oxidation or reduction process, an exchange of electrons, or a change in oxidative state of the molecule which have incorporated oxygen or other additives because the process does not proceed via a typical reduction-oxidation mechanism.

In the case of water containing a biological contaminant, the target molecule may be a portion of a biomolecule which is itself a contaminant present in the water, such as a protein, polysaccharide, or polynucleotide, or a portion of a biomolecule, such as a protein, DNA, or RNA, located within an infectious agent or organism contaminating the water. Energy is introduced at a frequency and intensity to selectively dissociate one or more target bonds in the target molecule, causing the target molecule to dissociate into component products compositionally distinct from the target molecule. Generally, the bonds of the target molecule do not reform because the components are no longer reactive with each other. In the case of biological contaminants such as infectious agent and organisms, the target molecule will preferably be a portion of a biomolecule essential for the function and/or survival of the infectious agent or organism. By selectively dissociating one or more target bonds in the target molecule, the infectious agent or organism is selectively killed or rendered inactive using the method. In certain embodiments, sample containing one or more biological contaminants is sterilized using the method.

In the case of air, the contaminant may include, but is not limited to, particulate matter, ozone, carbon monoxide, sulfur dioxide, nitrogen dioxide, and combinations thereof. In the case of such contaminants, the target molecule will preferably be the bonds present in particulate matter, ozone, carbon monoxide, sulfur dioxide, nitrogen dioxide, and combinations thereof. By selectively dissociating one or more target bonds in the target molecule, the contaminant is rendered inactive using the method. In certain embodiments, air sample containing one or more contaminants is sterilized using the method.

By irradiating a contaminated liquid and/or gas sample with energy at specific frequencies and intensities, target molecules can be selectively dissociated in a complex mixture. In some embodiments, the liquid and/or gas is irradiated with energy at multiple discrete frequencies and intensities in order to selectively dissociate one or more bonds within multiple different target molecules. For example, a water sample can be irradiated to simultaneously eliminate both chemical pollutants and biological contaminants in a water sample.

In certain embodiments, the system can be used to effectively eliminate the chemical pollutants, biological contaminants, and combinations thereof present in one or more contaminated liquids and/or gases without generating intermediates or by-products which require further remediation. The method can further include purification, for example, to remove the resultant component products or remove any catalyst used, if present.

In some embodiments, the liquid and/or gas can be irradiated by energy in the absence or presence of one or more catalysts, which may be dispersed throughout the liquid and/or gas or immobilized on a heterogeneous support present in the PES component of the system.

In certain embodiments, the method effectively eliminates chemical pollutants and biological contaminants in liquid and/or gas without generating intermediates or byproducts which require further remediation. In certain embodiments, the method effectively eliminates chemical pollutants and biological contaminants in water without requiring the addition of chemical reagents and/or heating or cooling the water during remediation.

Target Molecules

The methods described herein are used to dissociate one or more bonds in almost any molecule, permitting the remediation of virtually any chemical and/or biological contaminant.

Target molecules must contain at least one bond to be dissociated. Target molecules can be any compound which is a liquid and/or gas contaminant or a portion of a liquid and/or gas contaminant. Target molecules can be charged or uncharged. Target molecules can be naturally occurring or synthetically prepared compounds.

In some cases, the target molecule is a chemical contaminant, such as volatile organic compound, present in the sample. Examples of chemical contaminants that can be targeted using the methods described herein include alkyl sulfonates, alkyl phenols, ammonia, benzoic acid, carbon monoxide, carbon dioxide, chlorofluorocarbons, dioxin, fumaric acid, grease, herbicides, hydrochloric acid, hydrogen cyanide, hydrogen sulfide, formaldehyde, methane, nitrogenous wastes (e.g., sewage, waste water, and agricultural runoff), nitric acid, nitrogen dioxide, particulate matter, ozone, pesticides, polychlorinated biphenyls, oil, ozone, sulfur dioxide, and sulfuric acid.

Target molecules can be reactive or volatile aliphatic or aromatic organic compounds. In certain embodiments, the target molecule is a low-molecular weight organic compound, such as an alcohol, ester, ether, aldehyde, thiol, carboxylic acid, amine, amide, or ketone. The target molecule can also be a pharmaceutically active compound, or metabolite thereof.

In the case of samples containing a biological contaminant, the target molecule may be a portion of a biomolecule which is itself a contaminant present in the water, such as a protein, polysaccharide, or polynucleotide. The target molecule may also be a portion of a biomolecule, such as a protein, DNA, or RNA, located within an infectious agent or organism contaminating the sample. In the case of biological contaminants such as infectious agents and organisms, the target molecule will preferably be a portion of a biomolecule essential for the function and/or survival of the infectious agent or organism. By selectively dissociating one or more target bonds in the target molecule, the infectious agent or organism is selectively killed or rendered inactive using the method.

In some embodiments, the sample contains exclusively target molecules. In other embodiments, one or more target molecules are present in a sample with one or more non-target molecules. In these cases, the one or more target molecules can be selectively dissociated in a complex mixture. By way of illustration, in the case of a sample containing ammonia (a target molecule) in water, the method is used to selectively dissociate ammonia into N₂ and H₂ without dissociating water molecules into O₂ and H₂. In this case, water is not dissociated because the sample is irradiated with energy having the intensity and frequency required to dissociate the N—H bonds of ammonia and not the O—H bonds of water.

Target Bond

A target bond is any bond within a target molecule. Types of bonds affected by the dissociative process described herein include covalent, ionic, van der Waals, hydrogen bonding, or London dispersion forces or any bond which can form and has dissociation energy or energies if applied will break the bond and not allow the reformation of the bond.

Generally, the target bond is a covalent bond. The covalent bond can be a single bond, double bond, or triple bond. A non-limiting list of exemplary target bonds include N—H, C—H, C—C, C═C, C≡C, C—N, C═N, C≡N, C—O, C═O, C≡O, O—H, O—P, O═P, and C—X bonds, where X is any halogen selected from chlorine, fluorine, iodine, and bromine.

The energy of dissociation must be specific for the target bond of the target molecule. Bond dissociation energies are well known in the art. Examples of bond dissociation energies include H—H, 104.2 kcal/mol; B—F, 150 kcal/mol; C═C, 146 kcal/mol; C—C, 83 kcal/mol; B—O, 125 kcal/mol; N═N, 109 kcal/mol; N—N, 38.4 kcal/mol; C—N, 73 kcal/mol; O═O, 119 kcal/mol; O—O, 35 kcal/mol; N—CO, 86 kcal/mol; C═N, 147 kcal/mol; F—F, 36.6 kcal/mol; C—O, 85.5 kcal/mol; C═O (CO2), 192 kcal/mol; Si—Si, 52 kcal/mol; O—CO, 110 kcal/mol; C═O (aldehyde), 177 kcal/mol; P—P, 50 kcal/mol; C—S, 65 kcal/mol; C═O (ketone), 178 kcal/mol; S—S, 54 kcal/mol; C—F, 116 kcal/mol; C═O (ester), 179 kcal/mol; Cl—Cl, 58 kcal/mol; C—C, 181 kcal/mol; C═O (amide), 179 kcal/mol; Br—Br, 46 kcal/mol; C—Br, 68 kcal/mol C═O (halide), 177 kcal/mol; I—I, 36 kcal/mol; C—I, 51 kcal/mol; C═S (CS2), 138 kcal/mol; H—C, 99 kcal/mol; C—B, 90 kcal/mol; N═O (HONO), 143 kcal/mol; H—N, 93 kcal/mol; C—Si, 76 kcal/mol; P═O (POCl₃), 110 kcal/mol; H—O, 111 kcal/mol; C—P, 70 kcal/mol; P═S (PSCl₃), 70 kcal/mol; H—F, 135 kcal/mol; N—O, 55 kcal/mol; S═O (SO₂), 128 kcal/mol, H—Cl, 103 kcal/mol; S—O, 87 kcal/mol; S═O (DMSO), 93 kcal/mol; H—Br, 87.5 kcal/mol; Si—F, 135 kcal/mol; P═P, 84 kcal/mol; H—I, 71 kcal/mol; Si—Cl, 90 kcal/mol; PEP, 117 kcal/mol; H—B, 90 kcal/mol; Si—O, 110 kcal/mol; C═O, 258 kcal/mol; H—S, 81 kcal/mol; P—Cl, 79 kcal/mol; C≡C, 200 kcal/mol; H—Si, 75 kcal/mol; P—Br, 65 kcal/mol; N═N, 226 kcal/mol; H—P, 77 kcal/mol; P—O, 90 kcal/mol; C≡N, 213 kcal/mol.

In one embodiment, target bonds are dissociated heterolytically by the process described herein. When heterolytic cleavage occurs, ionic component products may be produced in addition to radicals and ejected electrons, for example:

A:B→A.+B⁺+e⁻, or

A:B→A⁺+B.+e⁻

The radicals can re-associate to form A:B, but in the preferred embodiment, the radicals re-associate in a homomeric fashion to form A:A and B:B component products. One, two, or more identical radicals can associate to form known ions, atoms, or molecules.

In some embodiments, target molecules contain multiple non-identical atoms, multiple oxidation states, or combinations thereof, all of which contain a variety of types of target bonds. Examples of target molecules with non-identical target bonds containing multiple non-identical atoms are dichloroethane (CH₂Cl₂) and ethanolamine (OHCH₂CH₂NH₂). Examples of target molecules with non-identical target bonds with multiple oxidation states include ethyl acetylene HC≡CH₂CH₃ and ethyl isocyanate (CH₃CH₂N═C═O).

In some embodiments, the target molecule is present in a range from 1 part per billion (ppb) or lower to very high concentrations. Those skilled in the art will recognize the energy of dissociation intensity and duration of energy of dissociation treatment will need to be adjusted based on concentration of target molecules in a sample. Higher concentrations of target molecules are successfully dissociated by increasing energy of dissociation power (wattage), increasing exposure time to the promoter, or a combination thereof.

Those skilled in the art will also recognize the energy of dissociation intensity and duration of energy of dissociation treatment will need to be adjusted based on the exposure time of the contaminated sample to the promoter.

### III. Portable Liquid and/or GAs Remediation with PES System

A. Mechanisms Related to Precise Energy Separation (PES)

An atom is ionized by absorbing a photon of energy equal to or higher than the ionization energy of the atom. Multiple photons below the ionization threshold of an atom may combine their energies to ionize an atom by a process known as multi-photon ionization. These concepts also apply to molecules. Resonance enhanced multi-photon ionization (REMPI) is a technique in which a molecule is subject to a single resonant or multi-photon frequency such that an electronically excited intermediate state is reached. A second photon or multi-photon then ejects the electronically excited electron and ionizes the molecule.

Among a mixture of molecules with different bond dissociation energies, selective activation of one chemical bond requires a mono-chromatic source. For example, in a compound containing N—H (bond dissociation energy of 3.9 eV) and C—H (bond dissociation energy of 4.3 eV) bonds, a specific photon source of 4.0 eV dissociates the N—H bond exclusively.

The process described herein relies on two main principles. The first principle is that the dissociation of target molecules requires breaking one or more bonds. Thus, a plurality of photons or other energetic sources are absorbed by a given molecule. The second principle is that dissociation of molecules in a complex mixture can be achieved with specific selection of the energy for dissociation (both frequency and intensity), defined herein as the promoter.

Energy of Dissociation and Energy Sources

The energy of dissociation is the energy required for dissociation of a target molecule, and is specific for the target bond or bonds within a target molecule. The energy of dissociation is tunable and specific for the bond dissociation energy of any target bond within any target molecule.

The energy of dissociation is applied at a frequency and intensity effective for both scission of the target bond and target molecule dissociation.

In an example, the target molecule is AB, and application of the energy of dissociation specific for the A-B bond results in ejection of an electron from the target bond yielding a radical, an ion, and an electron, according to the following possible mechanisms:

A:B→A.+B⁺+e⁻, or

A:B→A⁺+B.+e⁻

The ions and radicals can be stable isolable species, or can combine with other ions to form molecules, i.e. the component products. The ejected electrons can be captured by an electron sink. The intensity of the energy of dissociation must be such that re-association of components back into the target molecules does not occur.

In one embodiment, application of the energy of dissociation satisfies the bond dissociation energy of the target bond of a target molecule via a one step electronic process, and the target bond is dissociated. Once one target bond has been dissociated, the energy of dissociation source can be tuned to the frequency of a second target bond dissociation energy and applied to the sample to affect dissociation of a second target bond. The energy of dissociation sources can be tuned as needed to dissociate all target bonds of the target molecule. There are numerous apparatuses that can provide multi-energy or photons within a nano second or quicker to effect irreversible dissociation and prevent formation of reactants from the dissociated target molecule components.

In another embodiment, application of the energy of dissociation satisfies the bond dissociation energy of the target bond of a target molecule via a process involving the Rydberg excited state of the target molecule. First, the energy of dissociation source excites the target molecule to a Rydberg state, wherein the energy required to nearly remove an electron from the ionic core (the ionization or dissociation energy) of a target molecule has been achieved. Next, the same or different energy of dissociation source then supplies sufficient energy to eject the excited electron from the target bond. In this embodiment, one or more energy of dissociation sources can be used for each step. Once one target bond has been dissociated, the energy of dissociation source can be tuned to the frequency of a second target bond dissociation energy. The energy of dissociation sources can be tuned as needed to dissociate all target bonds of the target molecule.

For example, treatment of ammonia with an energy of dissociation occurs via the two-step process involving the Rydberg State. First, energy of dissociation treatment of 193 nm excites a shared electron in the N—H bond such that ammonia is in an excited Rydberg state. Subsequent energy of dissociation treatment of 214 nm energy expels the electron and dissociates ammonia into NH₂⁻ and H. •Subsequent dissociative processes will give component products which re-associate to form N₂ and H₂.

In one embodiment, the one-step process, the two-step process, or a combination thereof are used to dissociate the target molecule. In one embodiment, one or more energy of dissociation sources are used for dissociation of each target bond within a target molecule. In one embodiment, one or more energy of dissociation sources are used in combination for dissociation of each target bond within a target molecule.

An exemplary molecule contains N—H, C—O, and O—H bonds. The N—H bond is cleaved with application of a 193 nm and 214 nm xenon bulb energy of dissociation source. The C—O bonds are cleaved with a mono-chromatic pulse generator. The O—H bonds are cleaved with a combination of photocatalyst and UV radiation. All of these energy of dissociation sources comprise the energy of dissociation required for complete dissociation of all the bonds of the target molecule. In some cases this requires three or more bond energies to expel the electron. In some cases, a filter may be used to isolate wavelengths or energies from a wide range source.

Energy of Dissociation Sources

An energy of dissociation source in the PES component provides the energy of the promoter. The energy of dissociation source delivers irradiative energy, can initiate catalytic processes, or combinations thereof. An energy of dissociation source supplies electromagnetic energy, acoustic energy, or any other energy which meets the energy of bond dissociation required to dissociate the target bond. The type of energy of the dissociation source can be photonic, photo-catalytic, chemical, kinetic, potential, magnetic, thermal, gravitational, sound, light, elastic, DC or AC modulation current (electrical), plasma, ultrasound, piezoelectric, or electrochemical energy.

Energy of dissociation sources include any apparatus which can supply the specific bond dissociation energy to break target bonds of target molecules specifically without non-target molecule bonds being affected. Examples include mono-chromatic light, monotone sound, or any other mono-energy source.

In one embodiment, an energy of dissociation source is applied at the appropriate frequency and intensity to attain a multi-photon or multi-frequency energy of dissociation within a rapid time scale through use of a generator of nano to pico-pulse cycles.

In some embodiments, the energy of dissociation sources of the PES component can be or may contain frequency generators, electrical generators, pulse generators, plasma generators, arc lamps, pulse generators, amplifying generators, tunable lasers, pulse lamps, light emitting diodes, pulsed diodes, quantum dot-based diodes/lamps, ultraviolet lamps, ultraviolet lasers, pulse ultraviolet generators, and ultrasound generators.

In some embodiments, the energy of dissociation source is one or more reactor beds having any number of lamps, generators, and/or bulbs; lamps, generators, and/or bulbs having the same or different sizes in terms of diameter and length; lamps, generators, and/or bulbs having the same or different wattages and/or any combination of the foregoing. The lamps, generators, and/or bulbs useful in this method can be any shape, size, or wattage. For example, a pulse light source allows one to use a 10 watt input of energy and generate 400,000 watts of pulse energy within ⅓ of a second of output, thereby reducing energy usage and equipment size and cost.

In preferred embodiments, one or more energy of dissociation sources that are part of the PES system are a pulse tunable laser or diode, or lamp which are attached to a pulse generator and configured and coupled to the output of one or more energy storage units and powered by the energy storage units or energy storage banks of the system. The control module of the system can be used to controllably deliver stored power to the one or more energy of dissociation sources upon discharge of the power stored in the energy storage units. In certain embodiments, the plurality of supercapacitors or ultracapacitors present in the power generation component may be charged in parallel and discharged in series to effectively multiply the voltage and produce a high voltage discharge pulse and function as a Marx generator. High and/or pulsed voltages may be required depending on the particular selection of the energy of dissociation source(s). In some embodiments, high voltage is considered to be in the range of about 50 to about 50000 Volts, or higher as needed. In some embodiments, the plurality of supercapacitors or ultracapacitors present in the power generation component can be controllably fast charged and fast discharged based on the incorporation of a Pulse Forming Network (PFN).

A Pulse Forming Network can create ultra-fast pulses of intense energy (Power) from microsecond to nanosecond to attosecond pulses through pulse shaping and can provide coherent controlled molecular dissociation, as well as a multiphoton or energy packet ionization, which allows for lower energy photons to combine their energies so to dissociate a bond which has a higher energy of dissociation. By selecting only those specific energy frequencies or groups of frequencies and filtering out those frequencies which can produce by-products or can interfere with the desired bond dissociation, it is possible to precisely control the site for the dissociation. As a non-limiting example, it is possible to combine the energies of two 266 nm wavelength photons, with adjustments due to interference or shift, via an ultra-fast pulse process in order to break the hydrogen and oxygen bonds in water molecules, which has an energy of dissociation of 125 nm. Resonance enhanced multiphoton ionization, described above, and wave shaping, as well as PFNs and supercapacitors, are known by those skilled in the art.

Those skilled in the art will recognize that the nature of the target bond and target molecule will determine the identity, frequency, and intensity of energy of dissociation source required.

In one embodiment, photocatalytic processes may use ultraviolet light promoters, supplied by ultraviolet energy of dissociation sources that are positioned to emit photons of ultraviolet light. The ultraviolet light sources are generally adapted to produce light having one or more wavelengths within the ultraviolet portion of the electromagnetic spectrum. However, the method should be understood as including ultraviolet light sources that may produce other light having one or more wavelengths that are not within the ultraviolet portion (e.g., wavelengths greater than 400 nm) of the electromagnetic spectrum.

In other photocatalytic processes, the energy of dissociation source is replaced by other devices, such as lamps or bulbs other than ultraviolet fluorescent lamps or bulbs; non-ultraviolet light emitting diodes; waveguides that increase surface areas and direct ultraviolet light and any energy light source that activates a photocatalyst; mercury vapor lamps; xenon lamps; halogen lamps; combination gas lamps; and microwave sources to provide sufficient energy to the photocatalyst substance to cause the bond dissociation to occur.

In one embodiment, the photocatalyst is applied to the surface of a fiber optic device and activated from the inside by the specific energy of dissociation. The fiber optic device can be placed into a membrane through which air, solids or liquids flows.

Energy of Dissociation Source Intensity

Energy of dissociation source intensity is the quantity of energy supplied to the promoter, which treats a target molecule. Energy of dissociation source intensity is directly proportional to the number and percentage of bonds which can be dissociated. Low intensity energy of dissociation sources have the capability to dissociate a smaller proportion of target bonds compared to higher intensity energy of dissociation sources. For example, in a photonic energy of dissociation source, the greater the number of photons present, the higher the likelihood of ejecting electrons.

In one embodiment, energy of dissociation source intensity is increased by use of a pulse generator in conjunction with a lamp of the proper wavelength, or a tunable laser/diode. In a preferred embodiment, the pulse generator supplies a predetermined number of pulses per second.

Energy of Dissociation Source Frequency

The frequency of energy of the energy of dissociation source (in photonic cases, the wavelengths of radiant energy) specifically dissociates target bonds of target compounds. One frequency, multiple selected frequencies, or combinations of energy of dissociation source frequencies can be used depending on the chemical structure of the target material. The apparatus must deliver sufficient intensity of the dissociation energy to completely dissociate the bond in adequate numbers to satisfy the need of the end user.

Methods of determining the appropriate frequency at which a target bond can be dissociated is known in the art, and include resonance enhanced multi-photon ionization (REMPI) spectroscopy, resonance ionization spectroscopy (RIS), photofragment imaging, product imaging, velocity map imaging, three-dimensional ion imaging, centroiding, zero electron kinetic imaging (ZEKE), mass enhanced threshold ionization (MATI), and photo-induced Rydberg ionization (PIRI).

Wavelengths to dissociate hydrogens from ammonia are 193, 214, 222, 234 and 271 nm. Three or more of these wavelengths in combination break NH₃ into its components: N₂ (g) and H₂ (g) without producing ozone.

Examples of wavelengths for dissociation include 193 nm and 214 nm, both of which are required. A wavelength of 248 nm will break down Ozone. In a preferred embodiment, the energy of dissociation source frequency range is from 115 nm to 400 nm, with appropriate filters, to satisfy the precise frequency of dissociation energies required for hydrogen dissociation only. Adjustments are made for cage effect and molecular interaction.

In one embodiment, the energy of dissociation source frequency is supplied by a tunable laser or light energy source that subjects samples to a mono-energy.

If the proper dissociation bond energy at a sufficient intensity to dissociate a selected bond or group of bonds is applied, there are no indiscriminate or random molecules or atoms produced other than what will be determined by the selected bonds which are targeted for dissociation, eliminating the random production of undesirable by-products or intermediates seen in oxidation and reduction, or indiscriminate chemical reaction.

Catalysts

In one embodiment, the energy of dissociation source includes a catalyst. The catalyst enhances the rate of target bond dissociation. The catalyst can be any material of any physical configuration which is compatible with the sample and any other energy of dissociation sources. Catalysts may be monofunctional, multifunctional, or a combination thereof. Catalysts can be used alone or in combination with other catalysts. The catalyst is used to drive the reaction to 100% completion, i.e., dissociating generally every ammonia molecule into nitrogen and hydrogen. The catalyst can be used to lower the amount of power needed to drive the reaction to 100% completion. The catalyst is applied to the liquid and/or gas containing the target molecule or to an interface between the energy source and the target molecule wherein the target molecule contacts the catalyst. Catalyst is applied to a surface (such as a nanoparticle or tube), or dispersed into a liquid or suspension, through which the energy passes to the target molecules.

In a preferred embodiment, an energy of dissociation source includes a photocatalyst and photonic (light-based) energy source. The photocatalyst provides an effective means for converting light into chemical energy. The catalyst or photocatalyst is semi-conductive material such as titanium oxides, platinized titania, amorphous manganese oxide, and copper-doped manganese oxide, titanium dioxide, strontium titanate, barium titanate, sodium titanate, cadmium sulfide, zirconium dioxide, and iron oxide. Photocatalysts can also be semiconductors that support or are coated with a metal, such as platinum, palladium, rhodium, and ruthenium, or with strontium titanate, amorphous silicon, hydrogenated amorphous silicon, nitrogenated amorphous silicon, polycrystalline silicon, and germanium, and combinations thereof. Catalysts or photocatalysts can be 2-D and/or 3-D carbon-based materials such as graphene, graphene oxide, carbon nanotubes, reduced or partially reduced graphene oxide, graphite, bucky balls (i.e., C₆₀), as well as other carbon-doped semi-conductive or other magnetic materials, for example, graphene doped AMO. The carbon-based materials may also be used to support or may be coated with catalytic materials to form composites with catalytic or photocatalytic properties.

Other catalysts include Nickel-molybdenum-nitride, nickel-hydrogen (NiH₂ or Ni—H₂), Au—TiO₂, CdS, NaTaO₃, K₃Ta₃B₂O₁₂, Ga₈₂Zn₁₈)(N₈₂O₁₈), and Pt/TiO₂. Photocatalysts based on cobalt have been reported. Members are tris(bipyridine) cobalt(II), compounds of cobalt ligated to certain cyclic polyamines, and certain cobaloximes, cobalt(II)-hydride, indium tin oxide (ITO) anode, poly(3,4-ethylenedioxythiophene), catalysts made of cobalt (Co), nickel (Ni) and iron (Fe) elements, and titanium disilicide.

Catalysts may be modified to increase or optimize activity. Some of the parameters to increase activity include enhanced surface area, optimization of [Cu²⁺], and resultant morphology. The electronic properties of the catalyst may also be important since the AMO is mixed valence (Mn²⁺, Mn³⁺, Mn⁴⁺) and possible reduction of Cu²⁺ to Cu¹⁺. The most active photocatalysts can be analyzed with X-ray photoelectron spectroscopy to study the oxidation state of the copper in these materials. Catalysts are characterized with X-ray powder diffraction (XRD) to study any crystallinity of the materials, electron diffraction (ED) in a transmission electron microscope (TEM) to study both crystalline and amorphous content of the catalyst, and atomic absorption (AA) for compositions of the catalyst. Semi-quantitative analyses of the solid sample can be done by energy dispersive X-ray analyses in a scanning electron microscope (SEM).

### IV. PES-Based Treatment System

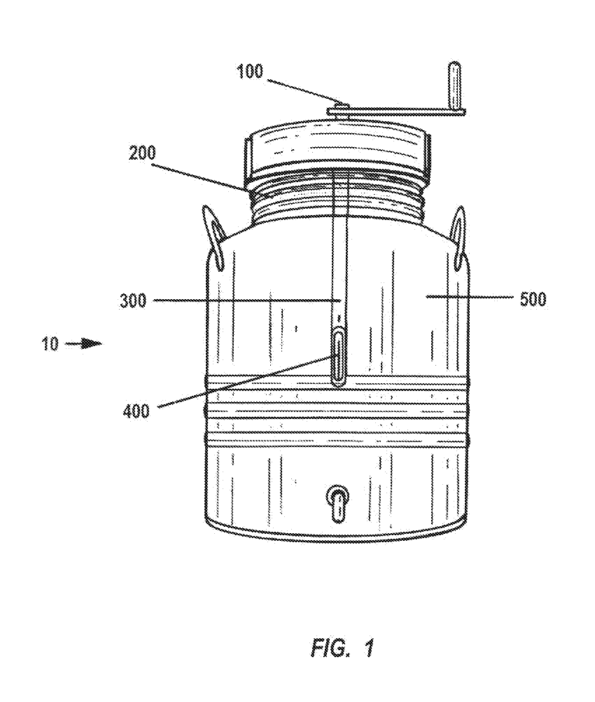

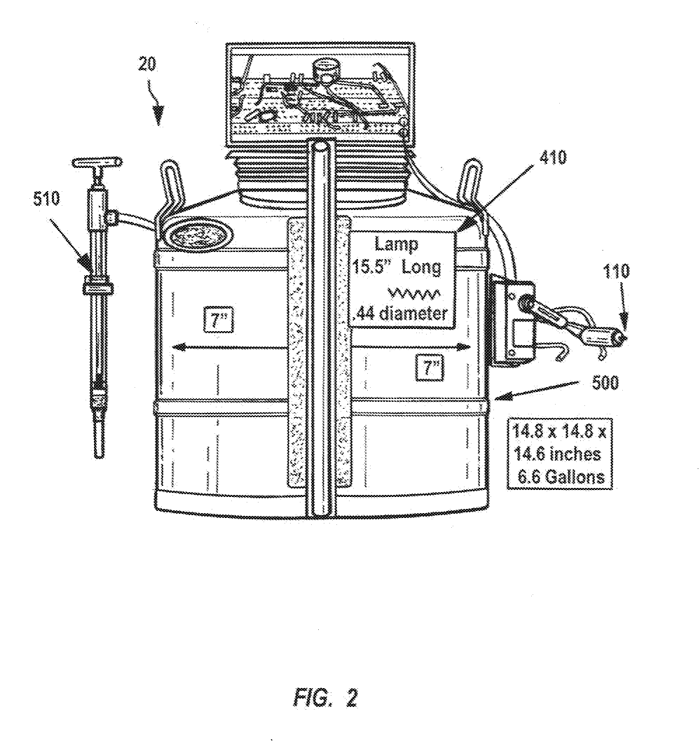

A system which includes a precise energy separation (PES) component and a power generation component is described herein. The following describes a prototype design of the system which can be modified or adapted, as necessary. Embodiments of the system are shown in FIGS. 1 and 2.

FIG. 1 shows a non-limiting example of a water treatment system as described herein. The system 10 includes a power generation component (100), energy storage component (200), PES component (300), and energy of dissociation source (400) and the components are housed in a main chamber/housing body (500).

It should be understood that the embodiments of the system are non-limiting and that a number of modifications could be made to the system and/or components which still fall within the scope of the system described herein. Furthermore, for the purposes of clarity, not every component is labeled in an illustration of the system and/or components. Nor is every component of each embodiment shown where illustration is not required to allow one of ordinary skill to understand the system and/or components.

Power Generation Component—100

One or more power generation source(s) are configured and coupled to a plurality of energy storage units and charge the units. The charging of the energy storage units is controlled via the control module. The various power generation components can include a dynamo, solar panels, turbines, fuel cells, optical rectennas, hydroelectric system, electrical generator, pulse generator, and other suitable power sources, as well as combinations comprising at least one of the foregoing power generation components.

In some embodiments, a direct current (DC) dynamo is used to generate power and the dynamo creates energy when it is cranked. FIG. 2 shows a non-limiting embodiment of the system which includes a dynamo with a hand crank 110. In other embodiments, the dynamo generates energy/power by spinning motion when cranked/rotated by a person, an animal, a water or wind driven turbine, or is rotated by any other suitable means. It is believed that the kinetic energy created by rotation is converted by the dynamo to useable power which can be used to charge the energy storage component. A dynamo may also be incorporated into a vehicle, such as a cart or a bicycle, as part of a wheel component such that motion of the vehicle rotates the dynamo and energy/power is generated. It should be understood that the one or more power generation sources may be obtained from commercial sources or may be constructed as needed using knowledge available in the art.

Energy Storage Component—200

The energy storage component contains a plurality of energy storage units as part of the system which are controlled by the control module during the charging process and the discharging process of the units.

Preferably, the energy storage units contain one or more supercapacitor or ultracapacitor cells. Although other types of energy storage units, such as batteries, may be used as well. It will be apparent to an individual skilled in the art that various types of super- or ultracapacitor cells may be used as components in the energy storage units of the system.

Super- or ultracapacitor cells used in the energy storage units may contain any suitable conductive material such as, but not limited to, activated carbon, graphite, carbon nanotubes, graphene, reduced or partially reduced graphene oxide, or carbon aerogels, which preferably have a high surface area (preferably greater than 200 m² g⁻¹). The conductive material may be optionally doped with one or more metal oxides (such as, for example, RuO₂, MnOₓ) to increase or improve the capacitive behavior/properties of the cells. The conductive materials are used in construction of active layers onto conductive metal electrodes (i.e. cathode, anode). The cells may be symmetric, having the same conductive material composition at each electrode or asymmetric, having different conductive material composition at each electrode. A suitable separator material is placed between the anode and cathode. Such separators include but are not limited to paper-based, filter paper, or commercially available type separators (i.e. Celgard). The super- or ultracapacitor cells additionally contain a suitable electrolyte. Electrolytes may be prepared in an aqueous or organic solvent, or may be an ionic liquid. Suitable aqueous electrolytes may be formed from acids (i.e. sulfuric acid, hydrochloric acid), alkali salts (i.e. sodium or potassium hydroxide), or other salts, such as phosphonium salts, sodium perchlorate, lithium perchlorate among others. Organic-based electrolytes may be prepared in solvents such as acetonitrile, propylene carbonate, tetrahydrofuran, and may contain a variety of ammonium or alkyl ammonium salts. Ionic liquid-based electrolytes, such imidazolium-based salts, among others may also be used. The gravimetric capacitance of the super- or ultracapacitors may be in the range of about 0.1 to about 1000 Farads per gram. One skilled in the art will understand that various types of super- or ultracapacitors architectures and selection of electrode, conductive materials, separator, and electrolytes may readily be performed based on knowledge available in the art to construct such capacitor cells. In some embodiments, the super- or ultracapacitors may be obtained directly from commercial sources. In some other embodiments, one or more commercially available capacitors may be used in place or in combination with the super- or ultracapacitors as described above. Conventional capacitors typically have capacitances which are in the range of about 0.01 to about 1000 microfarads.

In some embodiments, the system may contain two or more banks of energy storage units wherein each bank contains its own a plurality of super- or ultracapacitor cells. In a preferred embodiment, at least one energy capacitor-based storage bank is actively being charged, one or more banks is optionally in use and in active discharge to supply power to the PES component, and any remaining at least partially or fully charged banks may be in a stand-by reserve mode, at any given time.

The super- or ultracapacitor cells preferably possess or are selected to have appropriate dimensions to readily facilitate incorporation in the two or more energy banks of the system and are preferably modular for ease of replacement of each cell as needed.

In one embodiment, the at least one power generation source is configured and coupled to charge the super- or ultracapacitor cells of the energy storage units, which when at least partially or when fully charged can be discharged through the output which is configured and coupled to the PES component to provide power to the energy of dissociation source(s) of the PES system (i.e., xenon lamp or tunable laser). The use of supercapacitors or ultracapacitor cells in the energy storage units allows for excellent power delivery capabilities to the energy sources of the PES system, which may require high voltages and/or high pulsed voltages during operation. In some embodiments, high voltage is considered to be in the range of about 50 to about 50000 Volts, or higher as needed.

In certain embodiments, the plurality of supercapacitors or ultracapacitors of the power generation component are charged in parallel and discharged in series to effectively multiply the voltage and produce a high voltage discharge pulse. In such an embodiment, the energy storage units formed from a plurality of super- or ultracapacitors function as a Marx generator. In such a pulse generating system, the control module is used to control the charge and discharge cycle(s) of the supercapacitors.

Supplementary and/or Back Up Power

In certain embodiments, the one or more outputs of the energy storage units are optionally configured and coupled to provide energy stored by the energy storage units on discharge as a source of supplementary and/or back up to power to independent external systems, such as a home's electrical system or electronic devices, when the PES component described is preferably not in use.

Additional Components of Power Generation Component

In some embodiments, the power generation component and energy storage components can include one or more suitable sensors configured and coupled to one or more displays which can indicate the charging status and level of charge in the energy storage component.

Precise Energy Separation (PES) Component—300

The PES component of the system is configured and coupled to receive power stored in the energy storage units or banks of energy storage units. The PES component of the system includes one or more energy of dissociation sources, as described below, which can be used to irradiate contaminated liquids, such as water. Water can be treated by PES to afford treated water which has been rendered pure or purer than the water prior to treatment. Pure liquid typically refers to treated liquid that is at least 99.9% free of chemical, biological, and/or particulate-based agents, which are known pollutants. Purer liquid typically refers to treated water that is at least 90%, 91%, 92%, 93%, 94%, 95%, 96%, 97%, 98%, 99% free of chemical, biological, and/or particulate-based agents, which are known pollutants. In some other embodiments, the PES component of the system includes one or more energy of dissociation sources, can be used to irradiate one or more contaminated gases to afford treated gas or gases which have been rendered pure or purer than the gas(es) prior to treatment. Pure gas typically refers to treated water that is at least 99.9% free of chemical, biological, and/or particulate-based agents, which are known pollutants. Purer liquid typically refers to treated water that is at least 90%, 91%, 92%, 93%, 94%, 95%, 96%, 97%, 98%, 99% free of chemical, biological, and/or particulate-based agents, which are known pollutants.

Energy of Dissociation Source(s)—400

In preferred embodiments, the energy of dissociation sources of the PES component are pulsed light sources which typically include one or more band pass coatings applied to the interior of the quartz sleeves surrounding the light sources are used with quartz sleeves, for example, from Advanced Glass Industries and pulse generators, for example, from Applied Energetics. In some embodiments, xenon lamps/bulbs are used as the energy of dissociation source. In some embodiments, the xenon lamps/bulbs may be obtained from commercial sources. In some embodiments, the xenon lamps/bulbs are rated in the range of one to about 10000 Watts, more preferably ten to about 5000 Watts, and most preferably about 25 to about 2500 Watts. FIG. 2 shows a non-limiting embodiment of the system which includes a xenon lamp 410. The lights in their quartz sleeves are arranged in a set of concentric arrays in the disinfection chamber. The set of arrays should fit through the quick-release hatch, so that it can be quickly and easily removed for servicing (and easily installed). Each sleeve has its own light source (a Xenon pulse lamp and associated components). Preferred lights are 60-inch U-shaped, so that all the electronics need to be connected only at one side. 1600-J pulse bulbs are preferable utilized that can produce 1 W/cm² per second of liquid and/or gas flow. This output satisfies any standards the US or IMO set.

The quartz sleeves are no more than 4 inches apart (to protect against any light intensity loss due to turbidity). The liquid and/or gas moving through the disinfection chamber helps to dissipate the heat produced by the bulbs. Cooled nitrogen gas is constantly circulating inside the quartz sleeves that house the bulbs. The nitrogen flows into and out of each quartz sleeve to a central cooling chamber located outside of each sleeve. The nitrogen gas is cooled via a mini-heat exchanger and then returned to the sleeve. Aside from cooling, the nitrogen gas prevents formation of any oxidation products. The system needs a small circulation pump to circulate the nitrogen gas

The outside dimension of the U-shaped bulb is 3 inches, therefore the inside of the quartz sleeve must be at least 3.25 inches and the sleeve itself will be ¼ inch thick, making the overall size at least 3.75 inches. The cleaning wipers will be able to easily move back and forth to keep the sleeves clean.

An important parameter in the PES system design is the UV dose (UV intensity×exposure time), measured in mW/cm²—or in microW/cm² (1 mW/cm²=1,000 microW/cm²). Therefore, exposure or residence time has to be included as well as the lamp output being considered. Most important is UV transmittance (or absorbance). This is a measurement in a 1-cm path length cell of how much UV light is transmitted (or absorbed) by matter dissolved or suspended in the liquid, such as water. Distilled water is 100% pure and wastewater, dependent on treatment quality, can range from 20% to 80%. The more UV absorbed, the lower treatment per lamp. Table 1 below gives some approximate capacities per lamp at various water qualities to achieve <200 fecal coliforms per 100 ml.

TABLE 1
Requisite Lamp Capacities

| Water quality BOD/SS | % UV Transmission | Flow per lamp |
|---|---|---|
| (mg/L) | (1 cm) | (L/s) |
| 30-30 | 40 | 0.5-0.75 |
| 20-20 | 50 | 0.75-1.2 |
| 10-10 | 65 | 1-1.4 |

Each quartz sleeve is coated on the inside with a band gap filter coating that will permit only 254-nm light to pass through. The ends of the quartz sleeve are attached to a pulse generator, much like how a fluorescent light bulb is attached to its ballast. Applied Energetics sells a pulse generator that will activate 8-10 pulse bulbs.

Bulbs are preferably set to 3 pulses/second. The Xenon bulbs need to provide a range in the UV (185-280 nm). The internal coating will ensure that only 254-nm light is generated, which will break down the DNA/RNA in any living organisms (usually they will die outright, but even if they survive, they will no longer be able to reproduce).

The light source(s) and arrays are configured and coupled to the output of the power generation component and powered by the energy stored in the plurality of energy storage units/banks therein.

Sensors

In some embodiments, the PES component includes a suitable sensor that can detect whether the one or more energy of dissociation sources of the PES component are producing the needed intensity to provide a kill rate of 1 W/cm² per second of liquid and/or gas flow. In some embodiments, one or more sensors may be used which monitor and indicate the degree of sterilization of the liquid and/or gas during treatment. In some embodiments, sensors may be included which can indicate the status of the energy of dissociation source(s), such as for example, if the source(s) are operating within specified parameters. In one embodiment, a sensor may be included which indicates if the source(s) are overheating.

Additional Components of PES Component

In some embodiments, additional switches and control modules, can be included as necessary and used to control functions of the aforementioned PES or energy of dissociation sources.

Main Chamber/Housing Body—500

The PES-based component of the system can be used to treat varying amounts of contaminated liquids, such as water, and/or gases. As shown in FIG. 1, the overall system 10 includes a reaction chamber/housing which is preferably small enough to ensure ease of mobility and portability. The system 10 is typically cylinder-shaped with a diameter preferably in the range of about 5 to about 20 inches. The chamber which houses the PES component includes the one or more energy of dissociation sources, such as a lamp. The chamber may optionally include a filtration system to remove macroscopic contaminants, such as sediment, from the liquids or gases prior to or following remediation.

The system may be constructed from any suitable material such as a metal, metal alloy, or plastic which is stable and inert to the one or more energy of dissociation sources of the PES component. In some embodiments, the chamber/housing of the system is formed of 18/10 stainless steel. In some embodiments, suitable materials may include reflective coatings that are corrosion and abrasion-resistant.

In some embodiments, the volume of liquid or gases to be remediated in the chamber which may be treated at one time is in the range of about 0.1 to about 30 gallons, more preferably about 1 to about 15 gallons, and most preferably 5 to about 10 gallons. In some embodiments the liquid and/or gas may be introduced and removed from the chamber from a single opening. In certain other embodiments, the chamber may include two openings such that liquid and/or gas is introduced at one opening and removed through another. The openings used for introduction or removal of liquid and/or gas into the chamber of the system may each independently include a filtration system. The one or more openings of the system may include valves to control the flow of liquid and/or r gas being introduced or removed and/or caps needed to close the openings.

In preferred embodiments, the chamber may include one or more suitable mixing components, such as a paddle or stirring apparatus to create turbulence such that the liquid and/or gas is kept swirling around (turbulence) to ensure that all of the liquid and/or gas is treated by the one or more energy of dissociation sources of the PES component during treatment. The mixing components may be powered and driven by a motor, as needed. In some embodiments, the one or more mixing components which can be present in the system are connected and coupled to the power generation component and powered by the energy stored in the energy storage units. In some embodiments, the one or more mixing components are driven by

Optionally, liquid and/or gas to be treated may be passed through one or more pre-filter to remove large(r) macroscopic items present. In some embodiments, the liquid and/or gas flows first through one or more 50-micron filters of the filtration system upon entering or exiting the reaction chamber. In some embodiments, the system does include filtration capabilities and the liquid and/or gas is filtered separately by any suitable means prior to or following remediation using the system described herein.

Filtration Component

One or more pre-filters may be used to remove macroscopic solids from the liquid and/or gas to filter out particulate matter (≧50 μm). These filters are designed to be modular and removable for ease of replacement and cleaning, such as by washing or rinsing.

Additional Components of the Main Chamber/Housing Body

In some embodiments, additional sensors, switches, control modules, valves, spigots, or faucets may further be included as necessary and used to monitor and/or control functions of any of the aforementioned components including for liquid and/or gas filtration, liquid and/or gas mixing, release purified liquid and/or gas from the main chamber, and to determine the exposure during treatment necessary to remove or destroy contaminants.

FIG. 2 shows a non-limiting embodiment 20 of the system which includes a faucet/spigot component 510 present on the main chamber/housing body. In some embodiments, the filtration component described above may be incorporated into the faucet/spigot component.

Frequency of Use

A specific frequency of light at the proper intensity when applied to molecules, optionally in the presence of a catalytic or similar promoter, will dissociate any selected bond, resulting in the destruction or inactivation through atomic dissociation of the molecule. Accordingly, this method can be used to eliminate or inactivate biological contaminants, chemical contaminants, or combination thereof in contaminated liquid and/or gas. The component product gases, elements or chemicals can be purified, stored, utilized or disposed of.

In some embodiments, liquid and/or gas comprises target molecules is subjected to dissociation with an energy of dissociation to eliminate or inactivate one or more biological contaminants, chemical contaminants, or combination thereof. In preferred embodiments, the method effectively eliminates chemical pollutants, biological contaminants, and combinations thereof in a contaminated liquid and/or gas sample without generating intermediates or byproducts which require further remediation. The method can further include purification, for example, to remove the resultant component products or remove catalyst, if present. In certain embodiments, a sample containing one or more biological contaminants is sterilized using the method described herein.

Liquid and/or gas containing one or more chemical contaminants, biological contaminants, or combinations thereof is irradiated with energy at a frequency and intensity in an amount effective to selectively break one or more bonds within one or more target molecules. In doing so, one or more chemical contaminants, biological contaminants, or combinations thereof present in the liquid and/or gas are eliminated or otherwise rendered environmentally benign. Thus the liquid and/or gas treated using the system described herein is rendered a purified liquid and/or gas or a purer liquid and/or gas by application of PES to the contaminated liquid and/or gas.

In certain embodiments, the method effectively eliminates chemical pollutants and biological contaminants in one or more liquids and/or gases without generating intermediates or byproducts which require further remediation and/or without requiring the addition of chemical reagents.

Duration of the PES-Based Remediation Treatment

The PES-based remediation treatment is typically conducted for a period of time which is effective to remove all or a majority of target molecules which are contaminants and dissociate these into component products. Examples of duration of time include from about a fraction of a second up to about several hours, more preferably up to about one hour, most preferably up to about 30 minutes. In one embodiment, the remediation treatment is conducted for about one to about five minutes, more preferably about one to about two minutes.

Those skilled in the art will recognize the energy of dissociation source intensity, concentration of one or more contaminants, and energy of dissociation source energy required will affect the amount of time required for complete dissociation and remediation required.

Advantages of Portable Liquid and/or Gas Treatment System

A system as described herein which includes a Precise Energy Separation (PES) component and is powered by a power generation component can be used to remediate contaminated liquid and/or gas. The use of such a system is particularly advantageous for providing a means for remediating liquids, such as contaminated or polluted water, in remote regions of the planet where access to potable and drinkable water is limited. In some other exemplary uses of the system, it could also be used by recreational users/campers to purify water from sources such as, but not limited to rivers, lakes, or streams. The use of such a system is also particularly advantageous for providing a means for remediating gases, such as contaminated or polluted air, in remote regions of the planet where industrial gas contaminants and smog are a problem. In some other exemplary uses of the system, it could also be used by recreational users/campers to purify water from sources such as, but not limited to rivers, lakes, or streams.

The PES component of the system is powered by a power generation component which can contain one or more power generation source(s). The power generation source(s) generate power which is used to charge a plurality of energy storage units or banks of energy storage units, exemplary sources include a dynamo, solar panels, turbines, fuel cells, optical rectennas, hydroelectric system, electrical generator, pulse generator, and other suitable power sources, as well as combinations comprising at least one of the foregoing power sources.

The energy/power stored in the energy storage units or banks of energy storage units can be controllably delivered to and power one or more energy of dissociation sources present in the PES component of the system. The energy of dissociation source(s) can be used to expose a target pollutant to a burst of specific high intensity energy in order to dissociate it at the molecular level.

In some embodiments, the storage units or banks of energy storage units can be optionally configured and coupled to provide the energy/power stored therein to an electrical system, such as in a home, or used to power a lighting system or other devices. The energy/power stored by the energy storage units can be used as a source of supplementary and/or back up energy/power when the PES component of the system is preferably not in use.

The PES component of the system can consume up to 80% less energy than continuous wave mercury UV systems. Short, high energy bursts result in significantly less energy usage compared to the continuous mercury UV cycle. Additionally, PES selects only the wavelength or wavelengths which is needed to kill the invasive organism, thereby not producing unintended byproducts, or unknown photochemical reaction end products, such as oxidants or reducing chemicals as is seen with all electrochlorination systems, which can be more harmful than the original organism or chemical.

The PES component allows for tailoring or ‘tuning’ of key parameters including changing the peak power, pulse repetition rate, pulse sequencing, exposure duration, and wavelength or wavelengths (output energy). This unique flexibility helps in finding the optimal balance between high energy, low heat, short exposure times, and absolute kill rates.

PES can deliver high intensity bursts of energy in less than a second which results in higher throughput volumes for liquid and/or gas treatment.

In one embodiment, the PES component of the system uses xenon gas, eliminating the need for mercury gas-based system such as those used in standard UV systems. PES can, for example, select only 254 nm light which will disrupt the DNA of invasive species, thereby creating no oxidizing agents or harmful byproducts such as is found in current proposed systems. PES does not generate microwaves, nor does it contain mercury.

Certain water treatment options have a range of acceptable temperature ranges in which they are effective. For example, electrolytic disinfection, also known as electro-chlorination, cannot function below 5° C. or 41° F. This makes it impractical for use in extreme climates. In contrast, the PES component of the system described herein can operate equally effectively at any temperature on the surface of the Earth.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

I claim:

1. A portable system for treating one or more liquids, gases, surfaces, or combinations thereof comprising:
(a) a power generation component configured and coupled to power
(b) a precise energy separation (PES) component comprising at least one energy of dissociation source that irradiates the one or more liquids, gases, surfaces, or combinations thereof comprising one or more target molecules, the source providing an effective amount, intensity and frequency of energy to specifically dissociate one or more target bonds in the target molecules to separate the target molecules into their component products without producing any reaction by-products and without re-association of the one or more target bonds.

2. The system of claim 1, wherein the one or more liquids is water.

3. The system of claim 1, wherein the one or more gases is air.

4. The system of claim 1, wherein the power generation component comprises at least one power generation source configured and coupled to charge a plurality of energy storage units and an output configured and coupled to the PES component which receives power from the charged energy storage units upon discharge, and a control module.

5. The system of claim 4, wherein the at least one power generation source is selected from the group consisting of include a dynamo, solar panels, turbines, fuel cells, optical rectennas, hydroelectric system, electrical generator, pulse generator, and other suitable power sources, and combinations thereof.

6. The system of claim 4, wherein charging and discharging of the plurality of energy storage units is controlled by the control module.

7. The system of claim 6, wherein the plurality of energy storage units are contained in at least two or more separate banks of energy storage units.

8. The system of claim 7, wherein at least one of the banks of energy storage units is actively being charged, at least one of the banks of energy storage units is optionally in use and thereby in active discharge to supply power, and any remaining banks of energy storage units which are at least partially charged are in a stand-by reserve mode.

9. The system of claim 4, wherein the energy storage units comprise one or more supercapacitor or ultracapacitor cells.

10. The system of claim 9, wherein the supercapacitor or ultracapacitor cells are charged in parallel and discharged in series to produce a high voltage or pulsed voltage output upon active discharge.

11. The system of claim 4, wherein the power generation component further comprises an output which is configured and coupled to provide energy stored in the energy storage units on discharge and acts as a source of supplementary or back up power.

12. The system of claim 1, wherein the at least one energy of dissociation source of the PES system is powered by the power generation component.

13. The system of claim 12, wherein the energy of dissociation source is selected from the group consisting of frequency generators, electrical generators, plasma generators, arc lamps, pulse generators, amplifying generators, tunable lasers, pulse lamps, light emitting diodes, pulsed diodes, quantum dot-based diodes/lamps, ultraviolet lamps, ultraviolet lasers, pulse ultraviolet generators, ultrasound generators, and combinations thereof.

14. The system of claim 13, wherein the energy of dissociation source comprises energy selected from the group consisting of chemical, kinetic, potential, magnetic, thermal, sound, light, electrical, piezoelectric, electrochemical energy, and combinations thereof.

15. The system of claim 14, wherein the energy is in the form of light irradiation or electromagnetic radiation.

16. The system of claim 15, wherein the energy is amplified.

17. The system of claim 1, wherein the one or more liquids, gases, surfaces, or combinations thereof are irradiated in the absence of a catalyst.

18. The system of claim 1, wherein the one or more liquids, gases, surfaces, or combinations thereof are irradiated in the presence of a catalyst.

19. The system of claim 1, wherein the PES component comprises a catalyst.

20. The system of claim 19, wherein the catalyst is a semi-conductive material or magnetic material.

21. The system of claim 19, where in the catalyst is selected from the group consisting of titanium oxides (TiO₂), platinized titania, amorphous manganese oxide, copper-doped manganese oxide, titanium dioxide, strontium titanate, barium titanate, sodium titanate, cadmium sulfide, zirconium dioxide, and iron oxide.

22. The system of claim 19, wherein the catalyst is a semiconductor material selected from the group consisting of platinum, palladium, rhodium, and ruthenium, strontium titanate, amorphous silicon, hydrogenated amorphous silicon, nitrogenated amorphous silicon, polycrystalline silicon, germanium, and combinations thereof.

23. The system of claim 19, wherein the catalyst is selected from the group consisting of graphene or graphite, 2-D carbon-based material, 3-D carbon-based material, carbon-doped semi-conductive material, carbon-doped magnetic material, and combinations thereof.

24. The system of claim 1, wherein the one or more target molecules are a chemical contaminant.

25. The system of claim 24, wherein the chemical contaminant is selected from the group consisting of alkyl sulfonates, alkyl phenols, ammonia, benzoic acid, carbon monoxide, carbon dioxide, chlorofluorocarbons, dioxin, fumaric acid, grease, herbicides, hydrochloric acid, hydrogen cyanide, hydrogen sulfide, formaldehyde, medicines, methane, nitric acid, nitrogen dioxide, nitrates, nitrites, ozone, pesticides, polychlorinated biphenyls, oil, sulfur dioxide, sulfuric acid, volatile organic compounds, and combinations thereof.

26. The system of claim 1, wherein the one or more target molecules are a biological contaminant.

27. The system of claim 26, wherein the biological contaminant is selected from the group consisting of proteins, polysaccharides, polynucleotides, and combinations thereof.

28. The system of claim 26, wherein the biological contaminant is selected from the group consisting of bacteria, protozoa, viruses, plants, algae, plankton, animal cells, and combinations thereof.

29. The system of claim 26, wherein the target molecule is a portion of a biomolecule essential for the function and/or survival of the biological contaminant.

30. The system of claim 29, wherein the target molecule is selected from the group consisting of proteins, DNA, RNA, and combinations thereof.

31. The system of claim 1, wherein the energy of dissociation source of the PES component irradiates the one or more gases comprising the one or more target molecules which are a contaminant selected from the group consisting of particulate matter, ozone, carbon monoxide, sulfur dioxide, nitrogen dioxide, and combinations thereof.

29. The system of claim 1, wherein the one or more liquids, gases, or both is filtered prior to or following treatment.

30. The system of claim 1, wherein treatment of the one or more liquids, gases, or both is effective to render the one or more liquids, gases, or both pure or purer after treatment.

31. The system of claim 30, further comprising filtration of the one or more liquids, gases, or both to remove macroscopic contaminants
